(19) United States
(12) Patent Application Publication
(10) Pub. No.: US 2016/0045424 A1
(43) Pub. Date: Feb. 18, 2016

(54) COSMETIC FORMULATION CONTAINING COPOLYMER AND SULFOSUCCINATE AND/OR BIOSURFACTANT

(71) Applicants: Peter SCHWAB, Essen (DE); Uta KORTEMEIER, Essen (DE); Christian HARTUNG, (US); Ursula WESTERHOLT, Essen (DE); Silke LANGER, Düsseldorf (DE)

(72) Inventors: Peter SCHWAB, Essen (DE); Uta KORTEMEIER, Essen (DE); Christian HARTUNG, Essen (DE); Ursula WESTERHOLT, Essen (DE); Silke LANGER, Duesseldorf (DE)

(73) Assignee: EVONIK DEGUSSA GMBH, Essen (DE)

(21) Appl. No.: 14/783,142

(22) PCT Filed: Apr. 3, 2014
(86) PCT No.: PCT/EP2014/056637

§ 371 Date: Oct. 8, 2015

(30) Foreign Application Priority Data
Apr. 10, 2013 (DE) 102013206314.8

(51) Int. Cl. A61K 8/81; A61Q 5/00; A61Q 5/12; A61Q 19/10; A61K 8/60; A61Q 5/02

(52) U.S. Cl. CPC A61K 8/8152; A61K 8/8147; A61K 8/602; A61Q 5/02; A61Q 5/12; A61Q 19/10; A61Q 5/006; A61K 2800/30; A61K 2800/48

(57) ABSTRACT

The present invention relates to formulations comprising certain copolymers and at least one surfactant selected from the group consisting of sulphosuccinates and biosurfactants.

### FIELD OF THE INVENTION

The present invention relates to formulations comprising certain copolymers and at least one surfactant selected from the group consisting of sulphosuccinates and biosurfactants.

### PRIOR ART

A long-standing problem in the teaching of formulation of cosmetic compositions has been to achieve sufficiently high viscosities for formulations comprising sulphosuccinates or biosurfactants.

Carbomer thickeners for use in cosmetics are known; such carbomers are generally copolymers of (meth)acrylic acid and alkyl (meth)acrylates which have thickening properties when neutralised in aqueous solution, for example, with alkali. EP1953176 and EP 1950231 describe carbomers which have the capacity, unlike conventional carbomers, to provide neutralised solutions in the presence of salt which do not decrease in viscosity or transmission.

The object of the invention was to thicken cosmetic formulations comprising sulphosuccinates or biosurfactants.

### DESCRIPTION OF THE INVENTION

Surprisingly, it has been found that the cosmetic formulations described below and the use of the carbomers described are able to solve the problem addressed by the invention.

The present invention therefore relates to formulations comprising certain copolymers and at least one surfactant selected from the group consisting of sulphosuccinates and biosurfactants.

The invention further relates to the use of the particular copolymers for thickening cosmetic formulations comprising sulphosuccinates or biosurfactants.

An advantage of the present invention is that particularly mild, surfactant formulations are obtained.

A further advantage of the present invention is that the formulations do not leave behind any sticky skin sensation.

A further advantage of the present invention is that the formulations have good temperature stability, both in the warm and in the cold.

A further advantage of the invention is that the formulations have good flow properties.

A further advantage of the present invention is that the formulations have good foam quality.

A further advantage of the present invention is that the formulations have good foam stability.

A further advantage of the present invention is that the formulations foam rapidly.

A further advantage of the present invention is that the formulations work with a low WAS content and are therefore economically favourable.

A further advantage of the present invention is that the formulations readily moisten the skin.

A further advantage of the present invention is that the formulations are easily washed off.

A further advantage of the present invention is that the formulations may be prepared without heating.

A further advantage of the present invention is that the formulations are very reproducible, particularly with respect to the viscosity.

A further advantage of the present invention is that the formulations stabilise or solubilise insoluble additives, e.g. oil phases.

A further advantage of the present invention is that the formulations have very high transparency.

The present invention therefore provides cosmetic formulations comprising
A) at least one water-soluble copolymer having alkyl-modified carboxyl groups, obtainable by polymerisation of
   100 parts by weight of acrylic acid or methacrylic acid
   0.5 to 10 parts by weight of alkyl acrylate or alkyl methacrylate wherein the alkyl group is selected from alkyl groups having 12 to 24 carbon atoms and
   0 to 0.1, preferably 0.001 to 0.1, particularly preferably 0.001 to 0.044, parts by weight of a substance comprising two or more ethylenically unsaturated residues,
wherein a neutralised 1% by weight solution of the polymer in water has a viscosity of 1500 mPas or lower and a transmission of 90% or higher and this solution, on addition of 0.25 to 7 parts by weight of NaCl to 100 parts by weight of said solution, has a maximum viscosity of 10 000 to 40 000 mPas and a transmission of 90% or higher and
B) at least one surfactant selected from the group consisting of sulphosuccinates and biosurfactants.

In the context of the present invention, the term “neutralised” solution is understood to mean a solution of the polymer which has been adjusted to a pH of 7.0, measured at 25° C.

The viscosity specified in the context of the present invention is measured at 25° C. after one minute of rotation at a spin speed of 20 rpm using a BH type rotary viscometer and spindle no. 6.

The transmission specified in the context of the present invention is measured at a wavelength of 425 nm in a cuvette with an optical path of 1 cm after degassing the sample by centrifugation.

Within the context of the present invention, “biosurfactant” is understood to mean any glycolipid produced by fermentation.

Unless otherwise stated, all percentages (%) given are percentages by weight.

In a preferred embodiment, the formulation according to the invention comprises a component A), which is at least one water-soluble copolymer having alkyl-modified carboxyl groups, obtainable by polymerisation of
100 parts by weight of acrylic acid or methacrylic acid
0.5 to 5 parts by weight of alkyl acrylate or alkyl methacrylate wherein the alkyl group is selected from alkyl groups having 18 to 24 carbon atoms and
0 to 0.1, preferably 0.001 to 0.1, particularly preferably 0.001 to 0.044, parts by weight of a substance comprising two or more ethylenically unsaturated residues,
wherein a neutralised 1% by weight solution of the polymer in water has a viscosity of 1500 mPas or lower and a transmission of 90% or higher and this solution, on addition of 0.25 to 5 parts by weight of NaCl to 100 parts by weight of said solution, has a maximum viscosity of 15 000 to 40 000 mPas and a transmission of 90% or higher.

In an alternative preferred embodiment, the formulation according to the invention comprises a component A), which is at least one water-soluble copolymer having alkyl-modified carboxyl groups, obtainable by polymerisation of
100 parts by weight of acrylic acid or methacrylic acid
5 to 10, preferably 6 to 9.1, parts by weight of lauryl acrylate or lauryl methacrylate and
0 to 0.1, preferably 0.001 to 0.1, particularly preferably 0.001 to 0.044, parts by weight of a substance comprising two or more ethylenically unsaturated residues,
wherein a neutralised 1% by weight solution of the polymer in water has a viscosity of 1000 mPas or lower and a transmission of 95% or higher and this solution, on addition of 1 to 7 parts by weight of NaCl to 100 parts by weight of said solution, has a maximum viscosity of 10 000 to 30 000 mPas and a transmission of 95% or higher.

The copolymers present in the formulations according to the invention can be prepared by known methods; a detailed method description for preparing the copolymers present according to the invention is described in EP1953176 and EP 1950231.

Commercially available polymers have the names, for example, TEGO® Carbomer SER 841 (Evonik Industries), Aqupec SER W150C and Aqupec SER W300C (Sumitomo Seika)

Preferred formulations according to the invention are characterised in that the substance comprising two or more ethylenically unsaturated residues is selected from the group consisting of pentaerythritol allyl ether, diethylene glycol diallyl ether, polyethylene glycol diallyl ether and polyallyl sucrose.

The proportion of component A) in the formulation according to the invention is preferably from 0.01 to 10% by weight, preferably from 0.1 to 5% by weight and particularly preferably from 0.5 to 3% by weight, based on the total formulation.

The proportion of component B) in the formulation according to the invention is preferably from 0.1 to 30% by weight, preferably from 1 to 20% by weight and particularly preferably from 2 to 10% by weight, based on the total formulation.

The formulations according to the invention may comprise sulphosuccinates as component B). These include the salts of alkyl sulphosuccinic acid, also referred to as sulphosuccinic acid esters, and which include the monoesters and/or diesters of sulphosuccinic acid with alcohols, preferably fatty alcohols and particularly ethoxylated fatty alcohols. Preferred sulphosuccinates comprise C8-C18 fatty alcohol residues or mixtures thereof. Particularly preferred sulphosuccinates comprise a fatty alcohol residue which is derived from ethoxylated fatty alcohols. In this case, sulphosuccinates whose fatty alcohol residues are derived from ethoxylated fatty alcohols with a narrow homologue distribution are in turn particularly preferred. It is also possible to use alk(en)yl succinic acid preferably having 8 to 18 carbon atoms in the alk(en)yl chain or salts thereof. Particularly preferred sulphosuccinates are PEG- and sulphate-free. Examples of such PEG- and sulphate-free sulphosuccinates are the monosulphosuccinates of polyglyceryl-6 cocoate, polyglyceryl-4 caprate, polyglyceryl-4 caprylate, polyglyceryl-3 caprate or polyglyceryl-3 caprylate and also mixtures thereof. Particularly preferred PEG- and sulphate-free sulphosuccinates and preparation thereof are described in PCT/EP2013/050367.

The formulation according to the invention preferably has rhamnolipids, sophorolipids, glucose lipids, cellulose lipids and/or trehalose lipids, preferably rhamnolipids and/or sophorolipids, as component B) “biosurfactant”. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789 or the documents cited therein. Suitable biosurfactants can be acquired e.g. from Soliance, France.

The formulation according to the invention preferably has, as biosurfactants, rhamnolipids, in particular mono-, di- or polyrhamnolipids and/or sophorolipids. The formulation according to the invention particularly preferably has one or more of the rhamnolipids and/or sophorolipids described in EP 1 445 302 A with the formulae (I), (II) or (III).

A preferred formulation according to the invention is characterised in that said formulation comprises an additional component C), which is selected from the group of compounds containing ammonium, phosphonium, imidazolinium or imidazolium groups, preferably compounds containing quaternised ammonium groups, particularly preferably alkyl quats such as cetrimonium chloride (VARISOFT® 300), dialkyl quats or ester quats, or from the group of cationic polymers, preferably cationic guar, cationic siloxanes, and polyquaternium compounds, particularly polyquaternium 1 to polyquaternium 94, particularly preferably hydroxypropyl guar hydroxypropyltrimonium chloride, polyquaternium 7, polyquaternium 10 and polyquaternium 11, and silicone quaterniurn-22 (ABIL® T Quat 60 and ABIL ME 45).

In the context of the present invention, the term “compound containing ammonium, phosphonium, imidazolinium or imidazolium groups” includes compounds which, under the conditions of the given formulation, for example, due to the pH, predominantly have a proton on the aforementioned group and are therefore cationically charged. Such substances are preferably selected from the group of the aminosiloxanes, particularly preferably aminopropyl dimethicone (ABIL Soft AF 200) or amodimethicone.

The proportion of component C) in the formulation according to the invention is preferably from 0.05 to 30% by weight, preferably from 0.2 to 20% by weight and particularly preferably from 0.5 to 10% by weight, based on the total formulation.

Particular preference is given to formulations according to the invention which are essentially free from alkoxylated compounds. The term “essentially free from alkoxylated compounds” in connection with the present invention is to be understood to mean that the formulations have no notable amounts of alkoxylated compounds which exert a surface-active effect. This is particularly understood to mean that these compounds are present in amounts of less than 1% by weight, preferably less than 0.1% by weight, particularly preferably less than 0.01% by weight, based on the total formulation, in particular no detectable amounts.

Particular preference is given to formulations according to the invention which are essentially free from sulphates. The term “essentially free from sulphates” in connection with the present invention is to be understood to mean that the formulations have no notable amounts of sulphated organic compounds which exert a surface-active effect. This is particularly understood to mean that sulphated organic compounds are present in amounts of less than 1% by weight, preferably less than 0.1% by weight, particularly preferably less than 0.01% by weight, based on the total formulation, in particular no detectable amounts. Examples of sulphate-containing surfactants are sodium laureth sulphate, sodium lauryl sulphate, ammonium laureth sulphate, ammonium lauryl sulphate, sodium myreth sulphate, sodium coco sulphate, sodium trideceth sulphate or MIPA laureth sulphate.

Especially preferred formulations according to the invention are essentially sulphate-free and essentially free from alkoxylated compounds.

The present invention further relates to the use of the copolymers defined above as component A) for thickening cosmetic formulations comprising at least one surfactant selected from the group consisting of sulphosuccinates and biosurfactants. Preferred use according to the invention uses the preferred components A) mentioned above. The same applies to the formulations to be thickened in the context of the use.

The examples adduced below illustrate the present invention by way of example, without any intention of restricting the invention, the scope of application of which is apparent from the entirety of the description and the claims, to the embodiments specified in the examples.

### EXAMPLES

Values given in the Tables are % by weight based on the total formulation.

Thickening effect of TEGO® Carbomer SER 841 on difficult to thicken sulphosuccinate- and/or biosurfactant-containing formulations

The measured values in the following Table show that TEGO® Carbomer SER 841 is capable of thickening relevant formulations with lower salt content in the formulation, whereas Novethix® L-10 (INCI: acrylates/beheneth-25 methacrylate copolymer, [Novethix™ L-10 Polymer] manufactured by Lubrizol Advanced Materials, Inc.) either requires a lot of additional salt, or shows hardly any thickening effect.

| | TEGO ® | |
|---|---|---|
| | Carbomer SER | |
| | 841 | |
| PEG-free System 1 | 3040 mPas | not able to be |
| 4.8% REWOTERIC ® AM C, 32% | 0.3% NaCl | thickened |
| (Sodium Cocoamphoacetate) | 1% Carbomer | 1% Polymer |
| 4.8% CAPB, 38% | | |
| Cocamidopropyl betaine) | | |
| 3.6% REWOPOL ® SB F 12P, 95% | | |
| (Disodium Lauryl Sulphosuccinate) | | |
| PEG-free System 2 | 3170 mPas | 3210 mPas |
| 5.6% REWOTERIC ® AM C, 32% | 1.5% NaCl | 4.2% NaCl |
| (Sodium Cocoamphoacetate) | 0.1% Carbomer | 0.1% Polymer |
| 4.4% Lauryl Glucoside, 50% | | |
| 1.2% Coco Glucoside, 50% | | |
| 3.6% Sodium Disodium Cocoyl | | |
| Glutamate, 25% | | |
| PEG-free System 3 | 4100 mPas | 1710 mPas |
| 5.6% CAPB, 38% | 0% NaCl | 2% NaCl |
| 4.4% Lauryl Glucoside, 50% | 0.3% Carbomer | 0.3% Polymer |
| 1.2% Coco Glucoside, 50% | | |
| 3.6% REWOPOL ® SB F 12P, 95% | | |
| (Disodium Lauryl Sulphosuccinate) | | |

Thickening effect of TEGO® Carbomer SER 841 on difficult to thicken sulphosuccinate- and biosurfactant-containing formulations having positively charged polymers

The following Table shows that guar quat and polyquaternium 10 additionally enhance the thickening of a sulphosuccinate- and biosurfactant-containing formulation by the carbomer.

| | 1 | 2 | 3 | 4 | 5 |
|---|---|---|---|---|---|
| Lauryl Glucoside, 50% | 8.80 | 8.80 | 8.80 | 8.80 | 8.80 |
| Coco Glucoside, 50% | 2.30 | 2.30 | 2.30 | 2.30 | 2.30 |
| 3.6% REWOPOL ® | 3.80 | 3.80 | 3.80 | 3.80 | 3.80 |
| SB F 12P, 95% | | | | | |
| (Disodium Lauryl | | | | | |
| Sulphosuccinate) | | | | | |
| TEGO ® Betain F 50; | 14.70 | 14.70 | 14.70 | 14.70 | 14.70 |
| 38% (Cocamidopropyl | | | | | |
| Betaine) | | | | | |
| Hydroxypropyl guar | | 0.5 | 0.75 | 1 | |
| hydroxypropyltrimonium | | | | | |
| chloride | | | | | |
| Polyquaternium-10 | | | | | 1 |
| TEGO ® Carbomer 841 | 0.3 | 0.3 | 0.3 | 0.3 | 0.3 |
| SER | | | | | |
| Water | 70.1 | 69.6 | 69.35 | 69.1 | 69.1 |
| Viscosity | 5019 | 6837 | 8400 | 8900 | 5729 |
| pH | 5.7 | 5.7 | 5.7 | 5.7 | 5.7 |

Example formulations:

Mild Hair & Body Shampoo; PEG- and sulphate-free

| | |
|---|---|
| REWOTERIC ® AM C, 32% (Sodium | 27.30% |
| Cocoamphoacetate) | |
| ANTIL ® SPA 80 (Isostearamide MIPA; | 1.50% |
| Glyceryl Laurate) | |
| Water | 64.50% |
| TEGO ® Carbomer 841 SER | 1.00% |
| Sophorolipid (acid form) | 3.20% |
| NaCl | 1.50% |
| Preservative (Neolone PE) | 0.45% |
| Perfume (Powder Fruit) | 0.25% |
| Citric Acid (30%) | 1.50% |

Mild Hair & Body Shampoo; PEG-, sulphate- and betaine-free

| | |
|---|---|
| Plantacare 1200 UP, 50% (Lauryl | 8.80% |
| Glucoside) | |
| ANTIL ® Soft SC (Sorbitan | 0.60% |
| Sesquicaprylate) | |
| Water | 53.10% |
| TEGO ® Carbomer 841 SER | 0.50% |
| Plantacare 818 UP, 50% (Coco Glucoside) | 2.40% |
| Sodium/Disodium Cocoyl Glutamate; 25% | 14.40% |
| REWOTERIC ® AM C, 32% (Sodium | 17.50% |
| Cocoamphoacetate) | |
| Perfume (Powder Fruit) | 0.25% |
| Preservative (Neolone PE) | 0.45% |
| Citric Acid (30%) | 2.00% |

Mild Hair & Body Shampoo; PEG- and sulphate-free

| | |
|---|---|
| Plantacare 1200 UP, 50% (Lauryl | 11.70% |
| Glucoside) | |
| ANTIL ® Soft SC (Sorbitan | 0.50% |
| Sesquicaprylate) | |
| Water | 63.00% |
| TEGO ® Carbomer 841 SER | 0.50% |
| Plantacare 818 UP, 50% (Coco | 5.60% |
| Glucoside) | |
| TEGO ® Betain F 50, 38% | 18.00% |
| (Cocamidopropyl Betaine) | |
| Preservative (Neolone PE) | 0.45% |
| Perfume (Powder Fruit) | 0.25% |

PEG-, sulphate- and betaine-free Conditioning Shampoo

| | |
|---|---|
| 4.8% REWOTERIC ® AM C, 32% (Sodium | 27.30% |
| Cocoamphoacetate) | |
| ANTIL ® SPA 80 (Isostearamide MIPA; | 1.00% |
| Glyceryl Laurate) | |
| Water | 64.30% |
| TEGO ® Carbomer 841 SER | 0.50% |
| Disodium Polyglyceryl-3 Caprate/ | 3.20% |
| Caprylate Sulphosuccinate | |
| Preservative (Neolone PE) | 0.45% |
| Perfume (Powder Fruit) | 0.25% |
| Citric Acid (30%) | 3.00% |

Further formulations:

Mild Hair & Body Shampoo; PEG- and sulphate-free

| | |
|---|---|
| Plantacare 818 UP, 50% (Coco | 2.30% |
| Glucoside) | |
| TEGO ® Carbomer 841 SER | 1.50% |
| Water | 66.80% |
| Disodium Polyglyceryl-3 Caprate/ | 5.0% |
| Caprylate Sulphosuccinate | |
| REWOTERIC ® AM C, 32% (Sodium | 11.3% |
| Cocoamphoacetate) | |
| TEGO ® Betain F 50, 38% | 12.4% |
| (Cocamidopropyl Betaine) | |
| Preservative | 0.45% |
| Perfume | 0.25% |

Mild Hair & Body Shampoo; PEG- and sulphate-free

| | |
|---|---|
| TEGO ® Carbomer 841 SER | 1.10% |
| Water | 73.9% |
| Disodium Polyglyceryl-3 Caprate/ | 8.3% |
| Caprylate Sulphosuccinate | |
| REWOTERIC ® AM C; 32% (Sodium | 15.6% |
| Cocoamphoacetate) | |
| Preservative | 0.45% |
| Perfume | 0.25% |

Mild Hair & Body Shampoo; PEG- and sulphate-free

| | |
|---|---|
| TEGO ® Carbomer 841 SER | 0.50% |
| Water | 74.9% |
| Sodium Lauroyl Methyl Isethionate; 32% | 6.25% |
| Disodium Polyglyceryl-3 Caprate/ | 8.30% |
| Caprylate Sulphosuccinate | |
| REWOTERIC ® AM C; 32% (Sodium | 9.30% |
| Cocoamphoacetate) | |
| Preservative | 0.45% |
| Perfume | 0.25% |

Mild Hair & Body Shampoo; PEG- and sulphate-free

| | |
|---|---|
| TEGO ® Carbomer 841 SER | 1.50% |
| Water | 74.1% |
| TEGO ® Betain 810; 35.5% | 2.90% |
| (Capryl/Capramidopropyl Betaine) | |
| Disodium Polyglyceryl-3 Caprate/ | 8.30% |
| Caprylate Sulphosuccinate | |
| REWOTERIC ® AM C; 32% (Sodium | 12.50% |
| Cocoamphoacetate) | |
| Preservative | 0.45% |
| Perfume | 0.25% |

Body Wash

| | |
|---|---|
| TEGO ® Carbomer 841 SER | 0.20% |
| Water | 78.90% |
| Sodium Lauroyl Sarcosinate (30%) | 10.00% |
| TEGO ® Betain F 50; 38% | 10.00% |
| (Cocamidopropyl Betaine) | |
| Lactic Acid | 0.20% |
| Formic Acid | 0.70% |

Mild Hair & Body Shampoo

| | |
|---|---|
| REWOTERIC ® AM C; 32% (Sodium | 15.00% |
| Cocoamphoacetate) | |
| ANTIL ® Soft SC (Sorbitan | 0.90% |
| Sesquicaprylate) | |
| Perfume | 0.25% |
| Water | 66.20% |
| TEGO ® Carbomer 841 SER | 1.00% |
| Sophorolipid (acid form) | 3.20% |
| TEGO ® Betain F 50; 38% | 13.00% |
| (Cocamidopropyl Betaine) | |
| Preservative (Neolone PE) | 0.45% |

Mild Hair & Body Shampoo

| | |
|---|---|
| 4.8% REWOTERIC ® AM C, 32% (Sodium | 15.00% |
| Cocoamphoacetate) | |
| ANTIL ® Soft SC (Sorbitan | 0.90% |
| Sesquicaprylate) | |
| Perfume | 0.25% |
| Water | 66.20% |
| TEGO ® Carbomer 841 SER | 1.00% |
| Rhamnolipid | 3.20% |
| TEGO ® Betain F 50, 38% strength, Evonik | 13.00% |
| Industries AG | |
| Preservative (Neolone PE) | 0.45% |

Mild Intimate Wash Gel

| | |
|---|---|
| Water | 84.7% |
| TEGO ® Carbomer 841 SER | 1.10% |
| REWOPOL ® SB C 55; 54% (Disodium | 3.70% |
| PEG-5 Laurylcitrate Sulphosuccinate; | |
| Capryl/Capramidopropyl Betaine) | |
| REWOTERIC ® AM B U 185; 30% | 4.00% |
| (Undecylenamidopropyl Betaine) | |
| TEGO ® Betain 810; 35.5% | 2.00% |
| (Capryl/Capramidopropyl Betaine) | |
| LACTIL ® (Sodium Lactate; Sodium PCA; | 1.00% |
| Glycine; Fructose; Urea; Niacinamide; | |
| Inositol; Sodium Benzoate; Lactic Acid) | |
| Glycerol | 3.00% |
| NaOH (50%) | 0.50% |
| Preservative, Perfume | q.s. |

Mild Intimate Wash Gel

| | |
|---|---|
| Water | 84.7% |
| TEGO ® Carbomer 841 SER | 1.10% |
| Disodium Polyglyceryl-3 Caprate/ | 3.70% |
| Caprylate Sulphosuccinate | |
| REWOTERIC ® AM B U 185; 30% | 4.00% |
| (Undecylenamidopropyl Betaine) | |
| TEGO ® Betain 810; 35.5% | 2.00% |
| (Capryl/Capramidopropyl Betaine) | |
| LACTIL ® (Sodium Lactate; Sodium PCA; | 1.00% |
| Glycine; Fructose; Urea; Niacinamide; | |
| Inositol; Sodium Benzoate; Lactic Acid) | |
| Glycerol | 3.00% |
| NaOH (50%) | 0.50% |
| Preservative, Perfume | q.s. |

Creamy Anti-Dandruff Shampoo

| | |
|---|---|
| TEGIN ® G 1100 Pellets (Glycol | 3.00% |
| Distearate) | |
| Sodium Laureth Sulphate; 28% | 34.60% |
| REWOCID ® SB U 185; 50% (Disodium | 3.00% |
| Undecylenamido MEA-Sulphosuccinate) | |
| Perfume | 0.20% |
| Zinc-Pyrione NF (Zinc Pyrithione) (48%) | 2.00% |
| ABIL ® B 88183 (PEG/PPG-20/6 | 0.50% |
| Dimethicone) | |
| Water | 42.00% |
| TEGO ® Carbomer 841 SER | 0.20% |
| NaOH (25%) | 0.30% |
| REWOTERIC ® AM C; 32% (Sodium | 11.70% |
| Cocoamphoacetate) | |
| Citric Acid (30%) | 2.00% |
| ANTIL ® SPA 80 (Isostearamide MIPA; | 0.50% |
| Glyceryl Laurate) | |
| Preservative | q.s. |

Mild Shampoo

| | |
|---|---|
| Sodium Laureth Sulphate; 28% | 21.00% |
| Water | 61.30% |
| TEGO ® Carbomer 841 SER | 0.80% |
| REWOPOL ® SB FA 30 B; 40% (Disodium | 6.00% |
| Laureth Sulphosuccinate) | |
| TEGOSOFT ® LSE 65 K SOFT (Sucrose | 2.50% |
| Cocoate) | |
| TEGO ® Betain C 60; 47% | 5.90% |
| (Cocamidopropyl Betaine) | |
| VARISOFT ® 300 (Cetrimonium Chloride) | 2.50% |
| Preservative, Perfume | q.s. |

Body Wash

| | |
|---|---|
| ABIL Quat 3272, 50% strength in | 0.60% |
| propylene glycol | |
| TAGAT ® CH 40 (PEG-40 Hydrogenated | 0.50% |
| Castor Oil) | |
| Perfume | 0.30% |
| TEGO ® Betain 810; 35.5 | 2.00% |
| (Capryl/Capramidopropyl Betaine) | |
| Disodium Polyglyceryl-3 Caprate/ | 2.00% |
| Caprylate Sulphosuccinate | |
| Water | 91.00% |
| TEGO ® Carbomer 841 SER | 1.00% |
| TEGO ® Cosmo C 100 (Creatine) | 0.50% |
| TEGOCEL ® HPM 50 (Hydroxypropyl | 0.30% |
| Methylcellulose) | |
| VARISOFT ® 300; 30% (Cetrimonium | 1.30% |
| Chloride) | |
| LACTIL ® (Sodium Lactate; Sodium PCA; | 0.50% |
| Glycine; Fructose; Urea; Niacinamide; | |
| Inositol; Sodium Benzoate; Lactic Acid) | |
| Preservative, Perfume | q.s. |

Mild Baby Shampoo

| | |
|---|---|
| 4.8% REWOTERIC ® AM C, 32% (Sodium | 10.00% |
| Cocoamphoacetate) | |
| Disodium Polyglyceryl-3 Caprate/ | 5.00% |
| Caprylate Sulphosuccinate | |
| REWOPOL ® SB FA 30 B; 40% (Disodium | 10.00% |
| Laureth Sulphosuccinate) | |
| TEGOSOFT ® GC (PEG-7 Glyceryl | 0.50% |
| Cocoate) | |
| REWOMID ® IPP 240 (Cocamide MIPA) | 0.50% |
| VARISOFT ® PATC | 2.50% |
| (Palmitamidopropyltrimonium Chloride) | |
| Water | 70.00% |
| TEGO ® Carbomer 841 SER | 1.50% |
| Preservative, Perfume | q.s. |

| | 6 | 7 | 8 | 9 | 10 | 11 |
|---|---|---|---|---|---|---|
| Sodium Laureth | 32.0 | | | | | |
| Sulphate; 28% | | | | | | |
| TEGO ® Betain AB 1214; | | 10.0 | | | | |
| 30% (Coco Betaine) | | | | | | |
| Lauryl Glucoside; 50% | | | 18.0 | 6.0 | | |
| REWOTERIC ® AM C; | | | | | 28.0 | 19.0 |
| 32% (Sodium | | | | | | |
| Cocoamphoacetate) | | | | | | |
| Rhamnolipid; 76.9% | 4.0 | 11.5 | 4.0 | 11.5 | 4.0 | 8.0 |
| TEGO ® Carbomer 841 | 1.0 | 1.0 | 1.0 | 1.0 | 1.0 | 1.0 |
| SER | | | | | | |
| Water | 60.5 | 77.5 | 77.0 | 81.5 | 67.0 | 72.0 |
| Sodium Chloride | 2.5 | | | | | |
| Viscosity | 4800- | 3000- | 2300- | 2300- | 2600- | 3000- |
| | 9100 | 5400 | 4800 | 2900 | 8700 | 4400 |
| pH | 4-5 | 5.0 | 4-5 | 5.0 | 5-7 | 5.0 |

Body Wash

| | |
|---|---|
| Sodium Laureth Sulphate; 28% | 21.5% |
| Rhamnolipid; 76.9% | 8.0% |
| TEGO ® Carbomer 841 SER | 1.0% |
| Sodium Chloride | 2.5% |
| Water | 67.0% |
| Preservative, Perfume | q.s. |

Body Wash

| | |
|---|---|
| Sodium Laureth Sulphate; 28% | 10.5% |
| Rhamnolipid; 76.9% | 11.5% |
| TEGO ® Carbomer 841 SER | 1.0% |
| Sodium Chloride | 2.5% |
| Water | 74.5% |
| Preservative, Perfume | q.s. |

Body Wash

| | |
|---|---|
| TEGO ® Betain AB 1214; 30% (Coco | 30.0% |
| Betaine) | |
| Rhamnolipid; 76.9% | 4.0% |
| TEGO ® Carbomer 841 SER | 1.0% |
| Water | 65.0% |
| Preservative, Perfume | q.s. |

Body Wash

| | |
|---|---|
| TEGO ® Betain AB 1214; 30% (Coco | 20.0% |
| Betaine) | |
| Rhamnolipid; 76.9% | 8.0% |
| TEGO ® Carbomer 841 SER | 1.0% |
| Water | 71.0% |
| Preservative, Perfume | q.s. |

Body Wash

| | |
|---|---|
| Lauryl Glucoside; 50% | 12.0% |
| Rhamnolipid; 76.9% | 8.0% |
| TEGO ® Carbomer 841 SER | 1.0% |
| Water | 79.0% |
| Preservative, Perfume | q.s. |

Body Wash

| | |
|---|---|
| REWOTERIC ® AM C; 32% (Sodium | 9.5% |
| Cocoamphoacetate) | |
| Rhamnolipid; 76.9% | 11.5% |
| TEGO ® Carbomer 841 SER | 1.0% |
| Water | 78.0% |
| Preservative, Perfume | q.s. |

1. A cosmetic formulation comprising:
A) at least one water-soluble copolymer having alkyl-modified carboxyl groups, obtained by polymerizing:
100 parts by weight of acrylic acid or methacrylic acid,
0.5 to 10 parts by weight of alkyl acrylate or alkyl methacrylate wherein the alkyl group is selected from alkyl groups having 12 to 24 carbon atoms, and
0 to 0.1 parts by weight of a substance comprising two or more ethylenically unsaturated residues,
wherein a neutralized 1% by weight solution of the copolymer in water has a viscosity of 1000 mPas or lower, and a transmission of 90% or higher, and wherein the solution, on addition of 0.25 to 7 parts by weight of NaCl to 100 parts by weight of said solution, has a maximum viscosity of 10 000 to 40 000 mPas and a transmission of 90% or higher; and
B) at least one surfactant selected from the group consisting of sulphosuccinates and biosurfactants.

2. The cosmetic formulation according to claim 1, wherein
A) is at least one water-soluble copolymer having alkyl-modified carboxyl groups, obtained by polymerizing:
100 parts by weight of acrylic acid or methacrylic acid,
0.5 to 5 parts by weight of alkyl acrylate or alkyl methacrylate wherein the alkyl group is selected from alkyl groups having 18 to 24 carbon atoms, and
0 to 0.1 parts by weight of a substance comprising two or more ethylenically unsaturated residues,
wherein a neutralized 1% by weight solution of the copolymer in water has a viscosity of 1500 mPas or lower, and a transmission of 90% or higher, and wherein the solution, on addition of 0.25 to 5 parts by weight of NaCl to 100 parts by weight of said solution, has a maximum viscosity of 15 000 to 40 000 mPas and a transmission of 90% or higher.

3. The cosmetic formulation according to claim 1, wherein
A) is at least one water-soluble copolymer having alkyl-modified carboxyl groups, obtained by polymerizing:
100 parts by weight of acrylic acid or methacrylic acid,
5 to 10 parts by weight of lauryl acrylate or lauryl methacrylate, and
0 to 0.1 parts by weight of a substance comprising two or more ethylenically unsaturated residues,
wherein a neutralized 1% by weight solution of the copolymer in water has a viscosity of 1000 mPas or lower, and a transmission of 95% or higher, and wherein the solution, on addition of 1 to 7 parts by weight of NaCl to 100 parts by weight of said solution, has a maximum viscosity of 10 000 to 30 000 mPas and a transmission of 95% or higher.

4. The cosmetic formulation according to claim 1, wherein the substance comprising two or more ethylenically unsaturated residues is selected from the group consisting of pentaerythritol allyl ether, diethylene glycol diallyl ether, polyethylene glycol diallyl ether and polyallyl sucrose.

5. The cosmetic formulation according to claim 1, wherein A) is present in the formulation in a proportion from 0.01 to 10% by weight.

6. The cosmetic formulation according to claim 1, wherein B) is present in the formulation in a proportion from 0.1 to 30% by weight.

7. The cosmetic formulation according to claim 1, wherein the at least one surfactant is a biosurfactant, and the biosurfactant is selected from the group consisting of rhamnolipids, sophorolipids, glucose lipids, cellulose lipids and trehalose lipids.

8. The cosmetic formulation according to claim 1, further comprising at least one component C) selected from the group of:
compounds containing ammonium, phosphonium, imidazolinium or imidazolium groups, and cationic polymers.

9. The cosmetic formulation according to claim 8, wherein component C) is selected from the group consisting of hydroxypropyl guar hydroxypropyltrimonium chloride, polyquaternium 7, polyquaternium 10 and polyquaternium 11, and silicone quaternium-22.

10. The cosmetic formulation according to claim 1, wherein said formulation is essentially free from alkoxylated compounds.

11. The cosmetic formulation according to claim 1, wherein said formulation is essentially free from sulphates.

12. A method of thickening a cosmetic formulation comprising:
introducing at least one water-soluble copolymer having alkyl-modified carboxyl groups, obtained by polymerizing:
100 parts by weight of acrylic acid or methacrylic acid,
0.5 to 10 parts by weight of alkyl acrylate or alkyl methacrylate wherein the alkyl group is selected from alkyl groups having 12 to 24 carbon atoms, and
0 to 0.1 parts by weight of a substance comprising two or more ethylenically unsaturated residues,
wherein a neutralized 1% by weight solution of the copolymer in water has a viscosity of 1000 mPas or lower, and a transmission of 90% or higher, and wherein the solution, on addition of 0.25 to 7 parts by weight of NaCl to 100 parts by weight of said solution, has a maximum viscosity of 10 000 to 40 000 mPas and a transmission of 90% or higher, to a cosmetic formulation comprising at least one surfactant selected from the group consisting of sulphosuccinates and biosurfactants.
